(19) United States
(12) Patent Application Publication
(10) Pub. No.: US 2025/0313847 A1
(43) Pub. Date: Oct. 9, 2025

(54) APTAMER-BASED POLLUTANT PROTECTION

(71) Applicant: Worcester Polytechnic Institute, Worcester, MA (US)

(72) Inventors: Natalie G. Farny, Natick, MA (US); Yizhe Ma, Worcester, MA (US)

(21) Appl. No.: 19/093,457

(22) Filed: Mar. 28, 2025

Related U.S. Application Data
(63) Continuation-in-part of application No. 18/524,554, filed on Nov. 30, 2023.
(60) Provisional application No. 63/428,786, filed on Nov. 30, 2022.
(60) Provisional application No. 63/571,279, filed on Mar. 28, 2024.

(51) Int. Cl. C12N 15/115; A61P 39/00

(52) U.S. Cl. CPC C12N 15/115; A61P 39/00; C12N 2310/16; C12N 2310/3515; C12N 2320/32

(57) ABSTRACT

An aptamer having an affinity for contaminants or pollutants such as toxic metals and industrial chemicals may be introduced by a biocompatible delivery mechanism such as a DNA or RNA strand to which the aptamer is attached. The delivery mechanism delivers the aptamer, either as a direct nucleic acid sequence or expressed in a cell as a probiotic. When delivered as a prophylactic to the gastrointestinal tract (orally) as an aptamer or expressed within a probiotic cell, this would prevent absorption of metals and would thus reduce or eliminate the need for chelation therapy and thereby reduce disease burden. When used therapeutically, it could be ingested, or injected intravenously. Once bound, the toxic metals are expelled through normal gastrointestinal or urinary processes.

### RELATED APPLICATIONS

This patent application claims the benefit under 35 U.S.C. § 119 (e) of U.S. Provisional Patent App. No. 63/571,279, filed Mar. 28, 2024, entitled “APTAMER-BASED POLLUTANT PROTECTION,” and is a continuation-in-part (CIP) under 35 U.S.C. § 120 of U.S. patent application Ser. No. 18/524,554, filed Nov. 30, 2023, entitled “HEAVY METAL TOXICITY REMEDIATION,” which claims the benefit under 35 U.S.C. § 119 (e) of U.S. Provisional Patent App. No. 63/428,786, filed Nov. 30, 2022, entitled “HEAVY METAL TOXICITY REMEDIATION,” all incorporated herein by reference in entirety.

### REFERENCE TO AN ELECTRONIC LISTING

The contents of the electronic sequence listing WPI24-03 (W24-034-02) wipo_st26_listing.xml; Size: 10,242 bytes; and Date of Creation: Jul. 1, 2025) is herein incorporated by reference in its entirety.

### BACKGROUND

Earthen beings are subject to organic contaminants from the environment daily. Pollutant chemicals are abundant, such as in drinking water, in the soil used to grow food, accumulate in the food chain in fish and meat, and in the air we breathe. It is quite impossible for humans in a modern community to avoid them. For example, studies from the NIH(National Institute for Health) have shown that ˜93% of Americans have the metabolic products of bisphenol-A (BPA) in their urine. The health consequences of exposure to organic contaminants are not entirely known, however many of these chemicals are associated with the development of a range of cancers, elevated risks of diabetes and heart disease, risks to fetuses (birth defects, low birth weight), liver disease, immune disease, and others.

In one example, Lead (Pb(II)) poisoning remains a global public health problem, causing neurodevelopmental anomalies in children and increased risk of cardiovascular, renal, and neurological disease in adults. The current standard of treatment for lead toxicity is chelation therapy with oral medication, or EDTA (Ethylenediaminetetraacetic acid disodium salt dihydrate) chelation by intravenous administration. However, these therapies are typically only offered in the case of extremely high lead levels (blood lead levels above 45 ug/dL for children and 70 ug/dL for adults), even though much lower levels (3.5 ug/dL for children and 5 ug/dL for adults) are associated with negative health consequences. The neurotoxic effects of lead are permanent. Lead exposures are cumulative, and deposits into bones and can take years to be fully eliminated from the body.

### SUMMARY

A method of using short nucleic acid strands (DNA or RNA with any chemical base modifications) binds with and prevents absorption or ingestion of toxic organic pollutants such as Per- and Polyfluoroalkyl Substances (PFAS) (e.g. Perfluorooctanoic Acid, or PFOA), phthalates, dioxins, polycyclic aromatic hydrocarbons (PAHs) and bisphenols (e.g. BPA). While many strategies have been invented to remediate these contaminants from the environment, technology is lacking for individuals to protect themselves from acquiring these substances from their environment, via ingestion, absorption, or inhalation.

Conventional approaches attempt to remediate the exposure at the source, and prevent exposure to humans at the outset. This is impractical to scale, given the broad range of chemicals, exposure sources, and routes of exposure. There are no conventional approaches that focus on protecting the individual consumer, or providing therapy to a person who has a particularly elevated exposure to one or several of these pollutant chemicals.

Configurations herein provide a method of preventing absorption or ingestion of organic contaminants by selecting an aptamer based on an ability to bind and prevent absorption or ingestion of toxic organic pollutants, and introducing the selected aptamer into a body for binding and removal of a targeted pollutant. The beneficial aptamer can be introduced by injecting a modified nucleic acid including the selected aptamer into individuals to bind a toxin and eliminate it from the body, or expressing the selected aptamer as short RNA molecules within an engineered probiotic bacterial strain to be used as a probiotic in the gastrointestinal tract to absorb and eliminate a toxin and prevent it from being absorbed by the body.

In another example, an aptamer having an affinity for toxic metals such as lead is introduced by a biocompatible delivery mechanism such as a DNA or RNA strand to which the aptamer is attached. The delivery mechanism delivers the aptamer, either as a direct nucleic acid sequence or expressed in a cell as a probiotic. When delivered as a prophylactic to the gastrointestinal tract (orally) as an aptamer or expressed within a probiotic cell, the aptamer effectively prevents absorption of metals and would thus reduce or eliminate the need for chelation therapy and thereby reduce disease burden. When used therapeutically, it could be ingested, or injected intravenously. Once bound, the toxic metals are expelled through normal gastrointestinal or urinary processes.

Configurations herein are based, in part, on the observation that pollutants, such as PFAS and heavy metals such as lead are associated with negative health symptoms, and tend to cause cumulative negative effects that are problematic to reverse.

Unfortunately, conventional approaches to heavy metal toxicity suffer from the shortcoming that treatment is not pursued until already harmful levels have been absorbed, and because it is problematic to remove the absorbed toxins; rather, mere mitigation of additional intake is pursued. Accordingly, configurations herein substantially overcome the shortcomings of conventional approaches by providing an aptamer configured to bind with the pollutants, and deliver the aptamer to a patient physiology by DNA or RNA mediums, where the aptamer fragment is available to bind with the heavy metal, following which it is expelled as waste. Other metals, such as cadmium and copper, may also be sequestered.

Many lead exposures are via ingestion from environmental sources: water, food, or incidental ingestion of contaminated dust or paint chips. When the water or working conditions are to blame, it may be difficult or impossible to eliminate lead from the environment completely. The neurotoxic effects of lead are permanent, leading to lifelong cognitive deficits in these children and creating a disease burden that is borne disproportionately by racially diverse and low-income communities. Therefore, there is not only an urgent need but an environmental justice obligation to develop accessible and cost-effective methods to protect people from lead.

Additional uses can be derived according to configurations herein by designing a binding aptamer targeting a toxicity source such as presented by a toxic metal, and implementing the biocompatible delivery mechanism for introducing the binding aptamer.

In further detail, configurations herein, a method of prophylactic and therapeutic treatment of metal toxicity such as lead includes determining a binding aptamer having an affinity for a toxic metal, and generating a nucleic acid strand including the binding aptamer. The generated nucleic acid strand is delivered into a therapeutic region for binding and transport of the toxic metal, and subsequent elimination thought normal physiologic processes.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a treatment and remediation environment suitable for use with configurations herein;

FIG. 2 is a depiction of aptamer binding to a toxic metal for fluorescence based detection in the environment of FIG. 1;

FIG. 3 is a graph of mitigation of a toxic metal presence according to configurations herein;

FIG. 4 is a graph showing the efficacy of the Pb7S lead-binding DNA aptamer used as a prophylactic for reproductive toxicity of lead;

FIG. 5 is a graph showing the efficacy of the Pb7S lead-binding RNA aptamer used as a prophylactic for reproductive toxicity of lead;

FIG. 6 is a graph showing the efficacy of the Pb7S lead-binding DNA aptamer used as a prophylactic for neurotoxicity;

FIG. 7 is a process flow of delivery of an aptamer and introduction into a treatment regimen for binding with a toxic metal;

FIG. 8 shows an extension of the binding aptamer used as a prophylactic for hexavalent chromium (Cr(VI)) toxicity;

FIGS. 9A and 9B show a binding aptamer used for sequester of PFOA;

FIGS. 10A-10C show encoding or expressing the binding aptamer in a nucleic acid strand;

FIGS. 11A-11C show approaches for introducing the nucleic acid strand including the binding aptamer into a treatment region;

FIGS. 12A-12B show extraction of the target pollutant from the bound aptamer and delivery substance or organism; and

FIG. 13 shows qPCR (quantitative polymerase chain reaction) analysis of a binding aptamer expression in response to bisphenol A.

### DETAILED DESCRIPTION

Configurations herein are based, in part, on studies from the NIH(National institute for Health) have shown that substantial percentages of Americans have the metabolic products of bisphenol-A (BPA) in their urine. The health consequences of exposure to organic contaminants are not entirely known, however many of these chemicals are associated with the development of a range of cancers, elevated risks of diabetes and heart disease, risks to fetuses (birth defects, low birth weight), liver disease, immune disease, and more.

In conventional intervention strategies, a typical approach is to attempt to remediate the exposure at the source, and simply prevent an initial exposure to humans. Such an approach is unlikely to be effective or practical, given the broad range of chemicals, exposure sources, and routes of exposure. There is no conventional approach that focuses on protecting the individual consumer, or providing therapy to a person who has a particularly elevated exposure to one or several of these chemicals. The disclosed approach differs by developing nucleic acid-based technologies that would enable people to protect themselves against any contaminants to which they may be exposed. Such a proactive prophylactic approach not only guards against the unavoidable pollutant presence, but may also be employed therapeutically post-exposure.

In alternate configurations, heavy metal toxicity is associated with significantly increased risks of cancer and cardiovascular disease, affecting many millions of Americans, and hundreds of millions globally. Most adult humans have significant bioaccumulation of toxic metals (lead, cadmium, arsenic, and others). Despite the fact that we have ample scientific evidence to support the role of toxic metals in increased human disease risk, typically no medical intervention is taken for metal toxicity unless the exposure is so high as to be causing acute disease. The current standard of care for removing heavy metals from the human body is chelation therapy with EDTA disodium. This medication is delivered intravenously, and therefore requires medical supplies and professionals to deliver the treatment, typically in a doctor's office. Lead causes neurodevelopmental anomalies in children and increased risk of cardiovascular disease (CVD), renal damage, and neurological disease in adults Lead exposures are cumulative, and the neurologic damage caused is permanent. The molecular mechanism of lead toxicity is related to its ability to replace calcium in biological processes. Lead enters cells through calcium channels and can interfere with calcium ion flow, which is a central mechanism of its neurotoxic effects. Lead integrates into hydroxyapatite and can be stored for many years in bone, leaching back into the body even after exposures are eliminated and thus taking years or decades to be fully cleared from the body.

Aptamers are small DNA-or RNA-based oligonucleotides which are typically produced by the systematic evolution of ligands by exponential enrichment (SELEX) technology. Aptamers are short stretches of nucleic acids (<100 nucleotide single-stranded DNA or RNA molecules) that bind specifically to a target molecule or ion, and may include any chemically modified nucleic acid including but not limited to morpholinos, locked nucleic acids (LNAs), left handed (L-enantiomeric, also known as “mirror” DNA or RNA, or Spiegelmers) or including modifications such as but not limited to pseudouridines, 2′-F, 2′-OMe, 2′-MOE, phosphorothioated backbones, thio-phosphoroamidated backbones, or other modifications to increase stability, specificity, or immunotolerance.

Aptamers can be selected, through SELEX, to bind with high sensitivity and specificity to a target, which may be anything from an ion to a small molecule to a cellular protein or component. This binding can be used to sequester the target ion or molecule in the context of an engineered cell, a biofiltration surface, or a prophylactic or therapeutic delivered directly to an organism. However, aptamers generally do not encode for protein (like mRNA), or perform specific cellular functions (like tRNA or rRNA).

Under certain conditions, aptamers can fold into three-dimensional structures. Structural motifs within aptamers provide specific binding sites for small molecules or macromolecular compounds of several types, including cells, cell surface proteins, bacteria, and viruses; moreover, they interact with targets with high affinity and selectivity. Aptamers are sometimes referred to as chemical antibodies, but they have huge advantages over them, like increased stability, less expensive and less time-consuming production, ease of chemical modification, lower immunogenicity, and higher target range.

### Heavy Metals:

Certain aptamers, known as Pb7 and Pb14, have been shown to have low micromolar to high nanomolar affinity (1.60±0.16 UM and 0.76±0.18 μM, respectively) for lead ions and reported minimal cross-reactivity to other ions Structural analyses suggest that lead can associate with nucleic acids through so-called G-quadruplex (G4) structures. Most lead-binding aptamers have been developed for the purpose of lead detection in environmental and biological samples.

The affinity of aptamers to bind to lead in vitro has previously been shown for purposes such as water supplies. Conventional approaches do not address the prophylactic use of aptamers in an organism to prevent metal toxicity. Development of aptamer-based approaches to prevent gastrointestinal absorption of lead represents a significant improvement over reactive treatments such as chelation therapy, which cannot reverse lead-induced tissue damage. The application of nucleic acid aptamers to the prevention of heavy metal toxicity is innovative and has not previously been demonstrated.

Configurations herein demonstrate that an aptamer having an affinity for a particular metal toxin provides selective binding with the targeted metal toxin for subsequent elimination. The aptamer need only be delivered as a strand or fragment from a probiotic or other suitable biocompatible delivery mechanism. Configurations herein demonstrate effects on nematode Caenorhabditis elegans (C. elegans) which is a ˜1 mm transparent soil organism that has been commonly used as a laboratory model organism. The C. elegans model, demonstrates that that lead-chelating DNA and RNA aptamers applied in the presence of lead protect the animals from reproductive and behavioral toxicity. Both DNA and RNA versions of the aptamers are effective, and the protective effect is specific to lead and to the aptamer sequence. Similar approaches show that aptamers protect cultured cells from lead toxicity, and protect osteoblastic function. Such aptamer-based chelation can be further developed as a prophylactic or therapeutic strategy for human exposures to toxic metals by selecting an aptamer having an affinity for a specific, targeted toxic metal.

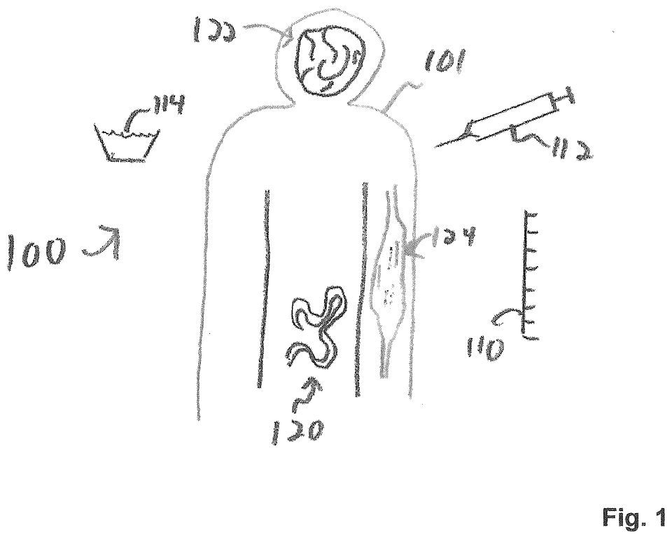

FIG. 1 is a context diagram of a treatment and remediation environment 100 suitable for use with configurations herein. In a patient 101 afflicted with toxic metal poisoning, the disclosed method of prophylactic and therapeutic treatment of metal toxicity includes determining a binding aptamer 110 having an affinity for a toxic metal, and generating a nucleic acid strand including the binding aptamer, such as a DNA or RNA for use as a biocompatible transport mechanism. An injected 112 or orally ingested 114 form of the generated nucleic acid strand is delivered into a therapeutic region for binding and transport of the toxic metal. This permits binding the binding aptamer to engage and bind with the toxic metal to form a bound aptamer, and expulsion of the bound aptamer via the urinary tract or gastrointestinal tract. For prophylactic treatment, the binding aptamer in the gastrointestinal region 120 binds with the toxic metal for excretion prior to any bodily absorption. For therapeutic treatment, presence of the nucleic acid strand with the binding aptamer 110 in the brain/central nervous system (CNS) 122 or musculature 124 will attract and bind to the toxic metal in tissue, and successive courses of treatment will tend to diffuse concentrations of the toxic metal out via the bloodstream, for example, and ultimately for extraction via the kidneys and urine.

One of the advantages of aptamers is the binding selectivity. Aptamers can be engineered to attract and bind specific targeted molecules. In the disclosed approach, lead remediation is a particularly beneficial approach, because lead tends to mimic calcium in human physiology, which facilitates migration into bones, in addition to other harmful anomalies. A binding aptamer is engineered that has a greater affinity for lead than for calcium, as it is important to not only expel the lead, but also to avoid collateral effects with normal biochemical processes. Potential binding aptamers may be selected to target a number of toxic metals, including but not limited to Pb, Cd, Co, Cr, Hg, Mn, Se, Fe, Ba, Be, Cs, Cu, Pt, Sb, Sn, Tl, V, Ni, U and W.

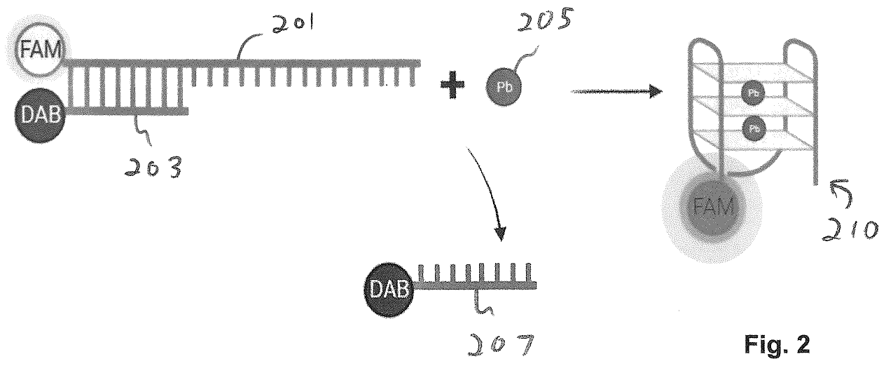

FIG. 2 is a depiction of aptamer binding to a toxic metal in the environment of FIG. 1. Referring to FIGS. 1 and 2, lead-binding single stranded (ss) DNA aptamers Pb7 and Pb14 (Table I) have been shown to have beneficial lead attraction potential. These aptamers were originally selected for use in a fluorescence-based detection assay for lead used for lead contamination in drinking water. The affinity for lead, or other toxic metals, can be leveraged by introducing a binding aptamer into a patient using a suitable biocompatible delivery mechanism, typically including a DNA or RNA based form in a controlled therapeutic or prophylactic approach. In the example of FIG. 2, the affinity of the binding aptamer is shown where the aptamers were 5′ end labeled with a fluor (fluorescein amidite, FAM) 201, and annealed to a shorter antisense strand with a 3′ quencher (dabcyl, DAB) 203, which quenched the fluorescent output. Upon aptamer binding to lead ions 205, the quench strand 207 was released, resulting in a fluorescent signal. In FIG. 2, 5′ FAM labeled aptamers are hybridized with short 3′ DAB labeled quench strands to form a partial double helix at the 5′ end of the aptamer. The proximity of the DAB to FAM quenches fluorescent signal. Upon addition of Pb(II) 205, the FAM-aptamer dissociates from the quench strand and forms a G quadruplex 210 structure around the lead ion, releasing the DAB-quench strand and resulting in detectable fluorescence.

TABLE I

| | Length | |
|---|---|---|
| Aptamer | (nt) | Sequence |
| Pb7 | 76 | (SEQ ID NO. 1) |
| | | GGAGGCTCTCGGGACGACGGCAGGGCTGTCG |
| | | TACGGTTTGTCGAAGGTGTCCCGATGCTGCA |
| | | ATCGTAAGAAT |
| Pb7S | 48 | (SEQ ID NO. 2) |
| | | GGGACGACGGCAGGGCTGTCGTACGGTTTGT |
| | | CGAAGGTGTCGTCCCGA |
| Pb7S | 48 | (SEQ ID NO. 3) |
| Antisense | | TCGGGACGACACCTTCGACAAACCGTACGAC |
| | | AGCCCTGCCGTCGTCCC |
| Pb7S | 48 | (SEQ ID NO. 4) |
| scrambled | | GCGGGCGATCTGCGGACGTTCTGAGCCTGAC |
| | | TGAGTGGGGACGCTGTA |

To confirm lead binding to the Pb7S aptamer, we reproduced the fluorescence-based lead binding assay, testing combinations of two flours (FAM and Yakima Yellow) and three quenchers (DAB, Black Hole Quencher 1 (BHQ1), and Iowa Black (IAB)). Using the Yakima Yellow fluor, we found a statistically significant difference in fluorescence from the no lead control at 20 μM lead, indicating an interaction of lead with the aptamer. The fluorescence detection system was highly sensitive to pH, with lower (pH 5.5) and higher (pH 8.4) values resulting in a loss of dynamic range, which was suspected as a caveat of using fluorescent detection, rather than a pH-dependent association of lead with the aptamer.

The conventional aptamer use incorporating fluorescence indicators covers detection only. Such a fluorescence labeling, quench strand, and the like are not used prophylactically or therapeutically. For human intervention, effective delivery vehicles as well as confirmation of no or merely acceptable side effects must also be established.

In a human or mammalian context, the delivery mechanism would deliver the aptamer in the form of DNA or RNA strand. The delivery mechanism to introduce the aptamer into the human physiology may be in the form of a capsule, therapeutic virus, probiotic bacteria, lipid nanoparticle, or other nanomaterials. The aptamer may be inside of the biocompatible delivery mechanism, or may be covalently or non-covalently attached to it. The aptamer may be released from the delivery mechanism or may remain within or bound to the delivery mechanism.

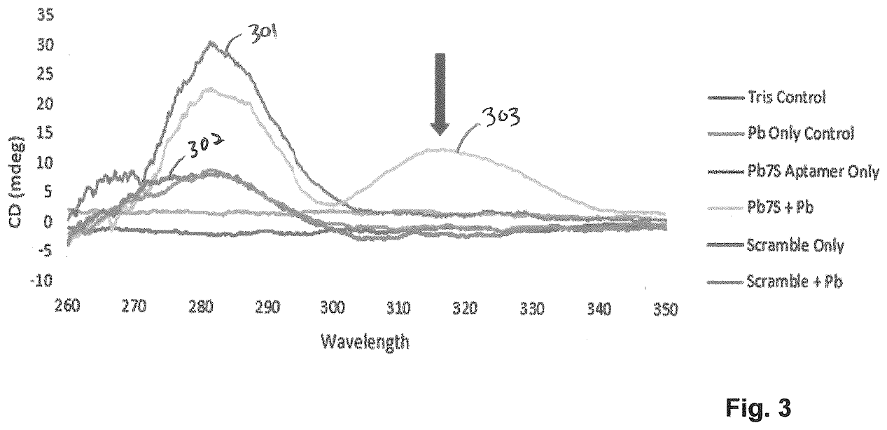

FIG. 3 is a graph of mitigation of a toxic metal presence according to configurations herein. Structural modeling predicts the formation of a G-quadruplex (G4) structure in the Pb7S aptamer. Lead ions are known to assemble into G4s with high affinity, creating unique G4 signatures by circular dichroism (CD) spectroscopy. To confirm the specific interaction of the Pb7S aptamer with lead ions in a manner independent from fluorescence detection, we applied CD to measure the lead-dependent assembly of the G4, shown in FIG. 3. The unbound Pb7S aptamer 301 and scrambled control 302 displayed a strong CD maximum in a single peak at 280 nm. The addition of lead ions to the Pb7S aptamer 303, but not the scrambled control, resulted in the concentration-dependent appearance of a broad peak with a maximum at 314 nm, reflective of the organization of a G4 structure. The formation of the peak at 314 nm was identical when the pH of the solution used was 5.5 or 8.4, suggesting that the interaction is not particularly sensitive to pH in this range. As lead mimics calcium within biological systems, we investigated the binding of the Pb7S aptamer to calcium, and found no evidence of G4 formation in response to calcium. Further, the presence of calcium did not alter the formation of the G4 structure when lead was added subsequently. From the above CD experiments, it can be concluded that lead ions bind with high specificity to the Pb7S aptamer through the formation of a G4 structure.

Referring again to the C. elegans experiments above, lead has previously been shown to result in reproductive toxicity in C. elegans, causing a dose-dependent decrease in brood size. These prior studies were conducted with animals exposed to metals by continuous growth in liquid cultures in multi-well plates. To better mimic dietary exposure to metals, we chose to expose our animals to metals by feeding. We first confirmed the dose-dependent decrease in brood size using our experimental feeding method. L3 stage animals were plated to NGM agar seeded with their food source OP50 E. coli mixed with lead acetate at concentrations from 0-25 mM. We found by this method that 15 mM lead exposure in the OP50 lawn was sufficient to cause an approximately 50% decrease in brood size.

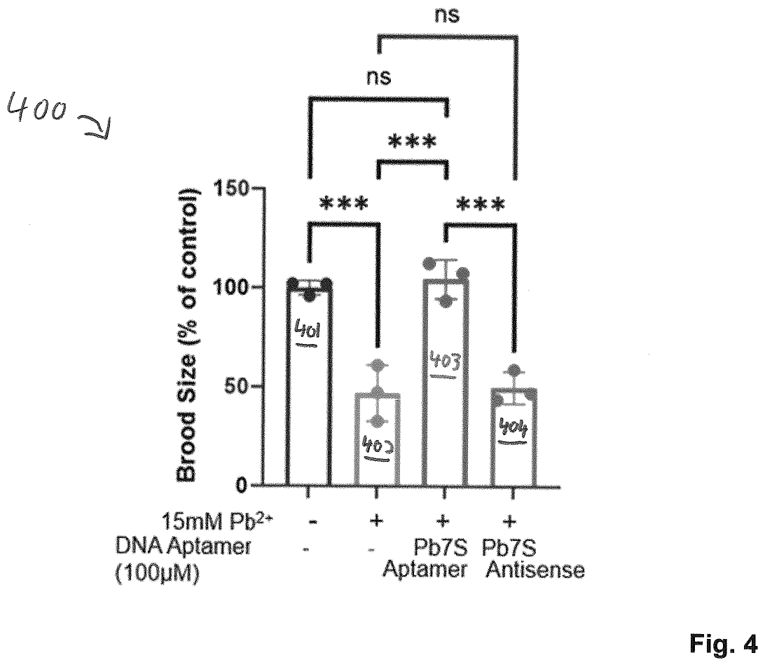

FIG. 4 is a graph 400 showing the efficacy of the Pb7S lead-binding DNA aptamer used as a prophylactic for reproductive toxicity of lead. To determine whether chelation of lead ions with aptamers could reduce reproductive toxicity, we employed three strategies to expose the animals to the aptamer: feeding, soaking, and drop casting. The feeding strategy mixed the aptamers at the designated concentration into the OP50, with or without lead, then animals were plated to this mixture and offspring were counted. The soaking strategy exposed animals to aptamers in an aqueous solution for 2.5 hours, then the animals were moved to NGM (Nematode Growth Medium) plates seeded with OP50 with or without lead. For the drop casting method, animals were plated to NGM plates containing OP50 with 402, 403, 404 or without 401 lead, then 10 μL of aptamer at the indicated concentration was dropped onto the animal. We observe by all methods (the drop cast method results are shown in FIG. 4) the Pb7S DNA aptamer result in protection of animals from lead-induced reproductive toxicity 403, whereas antisense (reverse complement strand DNA) controls 404 have no effect on brood size reduction caused by lead.

To thoroughly examine the protective effect of the Pb7S aptamer, the aptamer was tested at a range of both aptamer and lead concentrations. The minimum effective concentration of aptamer required to achieve full protection from exposure at 15 mM lead acetate was 2.5 μM. At 2.5 μM treatment, significant protection of animals was observed up to 100 mM lead acetate. Therefore, the results demonstrate the specific, dose-dependent protection of animals from ingested lead toxicity by exposure to lead-binding ssDNA aptamers.

Having determined an binding aptamer having an affinity for lead ions (human absorbed lead is typically Pb(II), or a Pb²⁺ ion form), and that favorable protection and extraction of lead was observed in laboratory trials, a delivery mechanism compatible with human physiology is called for. Modified RNAs (siRNAs and mRNAs) have been approved in the U.S. for therapeutic and prophylactic uses, and are a promising treatment modality.

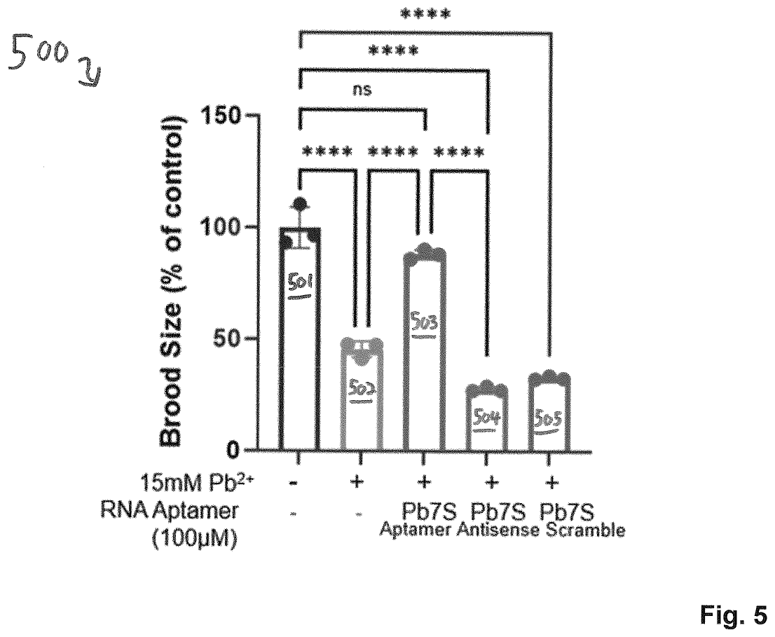

FIG. 5 is a graph 500 showing the efficacy of the Pb7S lead-binding RNA aptamer used as a prophylactic for reproductive toxicity of lead. To determine whether an RNA version of the Pb7S aptamer could also efficiently protect C. elegans from reproductive toxicity, we repeated our brood size assays using RNA versions of Pb7S and scrambled controls using the drop casting method. RNA aptamers 503 result in protection of animals from lead-induced reproductive toxicity, similar to lead negative samples 501, whereas scrambled 505 and antisense 504 controls have no effect on brood size. To examine the protective range of the RNA Pb7S aptamer, we tested the aptamer at a range of both aptamer and lead 502 concentrations. The minimum effective concentration of the RNA aptamer required to achieve full protection from exposure at 15 mM lead acetate was 2.5 μM. At 2.5 μM treatment, significant protection of animals was observed up to 100 mM lead acetate. These ranges were identical to those revealed in our ssDNA Pb7S aptamer testing. We conclude that ssRNA PB7S aptamers are equally as effective as ssDNA aptamers in protecting C. elegans from reproductive toxicity. Both DNA and RNA configurations can therefore be used for combining the nucleic acid strand with a biocompatible delivery mechanism for introduction into the subject patient.

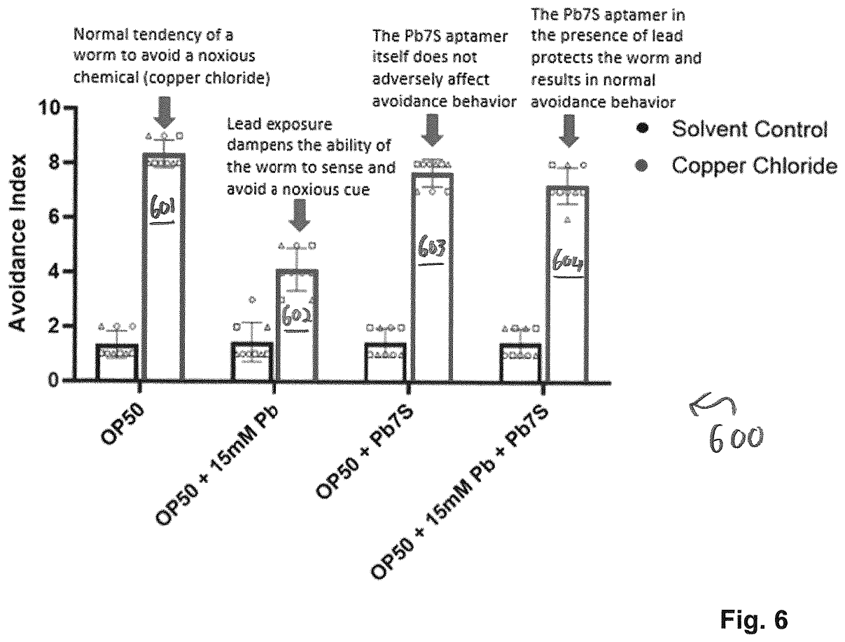

FIG. 6. is a graph 600 showing the efficacy of the Pb7S lead-binding DNA aptamer used as a prophylactic for neurotoxicity. Early lead exposure in children is well established to result in developmental neurotoxicity. We therefore sought to employ a model of developmental neurotoxicity in the form of a behavioral assay in our C. elegans model. C. elegans are known to move away from aversive cues, a pattern of behavior known as avoidance. To determine whether lead exposure negatively impacted C. elegans avoidance behavior during larval development, we exposed L1 stage worms to lead, then allowed them to develop to the L3/L4 stage in the presence of lead, and tested their avoidance of a noxious chemical cue. As shown in FIG. 6, lead exposure during larval development resulted in a dampened avoidance response to all noxious cues 602 as opposed to a normal avoidance response 601. Exposure to the Pb7S DNA aptamer in the absence of lead 603 had no effect on the normal avoidance behavior, suggesting the aptamer itself is not neurotoxic. When the animals were exposed to the Pb7S DNA aptamer in addition to the lead 604, there was a restoration of the normal avoidance behavior. The results suggest that the aptamer protects the animals from lead-induced neurotoxicity during development. The leftmost bar in each sample is a solvent control measure.

To determine whether ssDNA Pb7S aptamer could protect mammalian cells from lead toxicity, we used cell proliferation assays to measure the effect of lead on cultured cell growth. To utilize Pb7S in a human patient setting, a biocompatible vehicle needs to be generated for transporting the nucleic acid sequence including introducing the binding aptamer into a subject patient for remediation. Several approaches may be employed. An RNA therapeutic can be formed including the nucleic acid strand. Also, a probiotic approach can form a probiotic including the nucleic acid strand by appending the nucleic acid strain to a DNA strand; and replicating the DNA strand including the binding aptamer, Pb7S in the disclosed example. Then the DNA would be transcribed into multiple copies of an RNA aptamer for targeting the toxic metal. A suitable approach includes adding or editing the DNA of the probiotic, bacteria, or other biocompatible organism to contain the sequence of the aptamer strand.

As indicated above, the current standard of treatment for lead toxicity is chelation therapy with oral medication, or EDTA chelation by intravenous administration. However, these therapies are typically only offered in the case of extremely high lead levels (blood lead levels above 45 ug/dL for children and 70 ug/dL for adults), despite the fact that much lower levels are associated with negative health consequences, as discussed above. The recommended course of action for lower blood lead levels (3.5-45 ug/dL) is to continue to monitor the lead levels of the patient and attempt to identify and eliminate the source of contamination. Again, this course of action cannot reverse permanent neurologic damage, nor can it prevent the accumulation of lead in bones. Interventions to protect exposed individuals against low amounts of lead are lacking and are urgently needed.

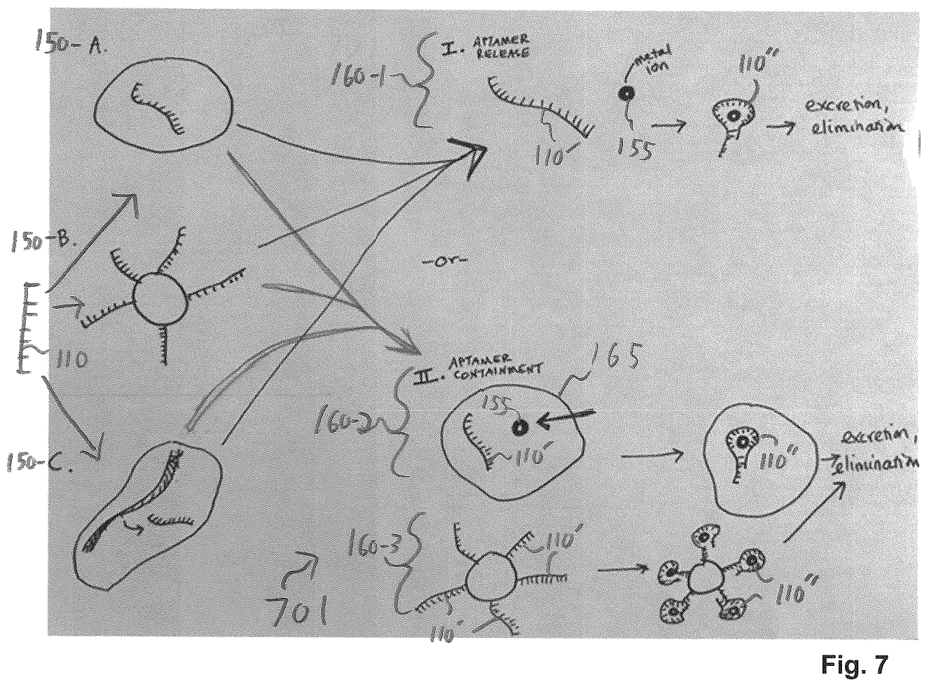

FIG. 7 is a process flow of the disclosed aptamer and introduction into a treatment regimen; and generating a biocompatible vehicle, such as a DNA or RNA or other suitable structure, to introduce the binding aptamer into a patient physiology. As disclosed above, the biocompatible delivery mechanism may be any suitable therapeutic virus, probiotic bacteria, lipid nanoparticle, or other nanomaterials. The delivery vehicle, while not exclusively RNA or DNA, would contain or deliver the aptamer, which includes the RNA or DNA. Referring to FIGS. 1 and 4, the binding aptamer 110 is generated, developed or identified to have an affinity for binding to a target toxin, such as lead. An editing or sequencing application is employed to form a biocompatible delivery vehicle 150, such as an RNA or DNA strand with the binding aptamer 110 included. The biocompatible delivery mechanism is employed for delivery of the binding aptamer 110 (aptamer). The binding aptamer may be inside of the biocompatible delivery mechanism 150-A, may be covalently or non-covalently attached to it 150-B or genetically expressed within a living delivery system such as a probiotic bacteria 150-C. For any delivery vehicle, the aptamer may be released 160-1 from the delivery vehicle or may remain within 160-2 or bound to 160-3 the delivery mechanism.

In general, the treatment involves a therapeutic compound with a nucleic acid strand including a binding aptamer, such that the binding aptamer has an affinity for a toxic metal, and a biocompatible delivery vehicle including at least one of a DNA or RNA structure, where the structure includes the binding aptamer. Any suitable biocompatible delivery mechanism may be employed. Various derivative or alternative DNA or RNA chemistries, included but not limited to ribose or deoxyribose sugar ring modifications (e.g., locked nucleic acids (LNAs), 2′-O-methyl, 2′-O-methoxyethyl), base substitutions (e.g., pseudouridine), left-handed or “mirror” DNA (L-DNA), backbone modifications (e.g., phosphorothioate (PS), Thiophosphoramidate, Morpholino), and glycosylated nucleic acids may be employed.

Whatever biocompatible delivery mechanism is employed, the binding aptamer may be appended to a strand of the biocompatible delivery vehicle, as an addition to a DNA or RNA strand, or may be in the form of a probiotic including cells 165 having DNA with a strand of the binding aptamer included in the DNA. Other suitable biocompatible delivery mechanisms may be employed for introducing the binding aptamer into a patient physiology, such as formulation into a lipid nanoparticle for injection, or encapsulation into a tablet or capsule for oral delivery (in addition to introduction by a probiotic bacterial or yeast strain).

Returning to FIG. 7, upon introduction into a patient physiology 701, the binding aptamer 110′ has an affinity for binding with the target toxin and binds or “wraps” around the target toxin 155, such as by a forming a G-quadraplex from the combination of the now-bound aptamer 110″ with the target toxin 155. Other suitable binding approaches may be employed, based on the selectivity and affinity of the binding aptamer to dissociate from a molecule defining the delivery mechanism 150 and form a bond with the target toxin. The bound toxin is then capable of removal by patient physiology as waste, via the kidneys or GI tract.

In the case of prophylactic measures, it is expected that a GI presence of the binding aptamer can eliminate lead prior to absorption into tissue. Subsequent to absorption, however, intravenous or tissue presence of the aptamer can still draw the target toxin from tissue based on the affinity and normal diffusion in a therapeutic approach. In the case of Pb7S and lead, the selectivity of the binding aptamer is such that beneficial calcium will not be targeted, even though the emulation of calcium by lead is a common result of lead poisoning.

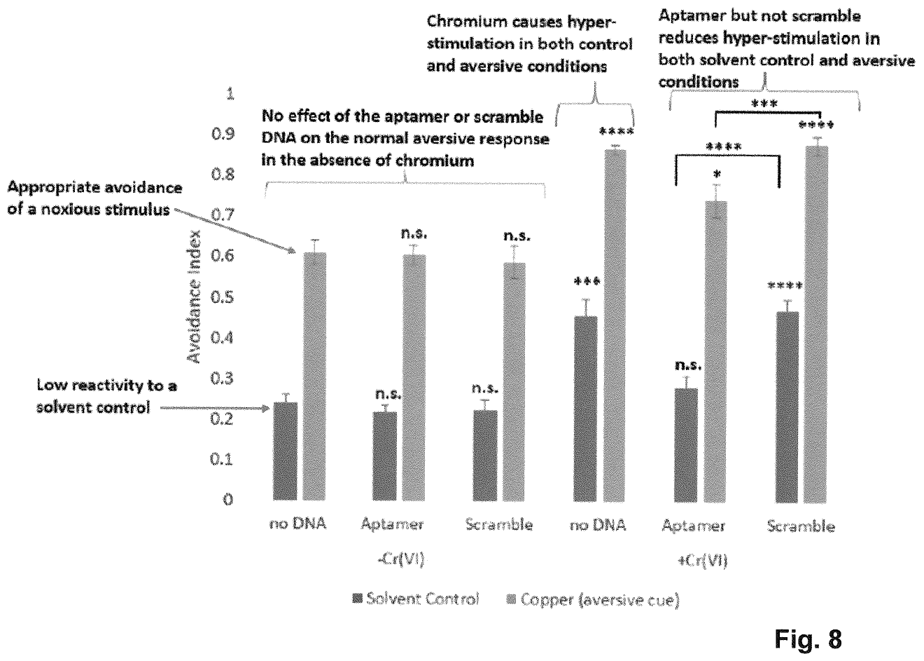

FIG. 8 shows an extension of the binding aptamer used as a prophylactic for hexavalent chromium (Cr(VI)) toxicity. The sequence is as follows:

(SEQ ID NO. 5)
5'CCACGCATAGGGCAAATCAAGCACACCCTCTAATGTTGCCTCTGATTC
TGGCCTATGCGTGC-3'

It has been shown that highly toxic Cr(VI) causes a hyper-stimulatory behavioral phenotype in the earthworm species Eisenia fetida (PMIDs: 15978294, 29621711). Using the same methods described in this proposal, we have confirmed: a) that Cr(VI) causes a hyper-stimulatory aversive behavioral response in C. elegans which is consistent with phenotypes observed in earthworms; and b) that Cr(VI)-binding aptamers, but not antisense or scrambled controls, prevent this hyper-stimulatory phenotype and provide protection against Cr(VI)-induced behavioral toxicity. The result demonstrates the protective action of aptamers in a context where the behavioral anomaly is distinct from that caused by Pb(II). This result is significant because it supports our claim that aptamer-based prophylactic strategies could be useful against a range of environmental toxicants that cause variable toxic phenotypes. Our results with lead-binding aptamers are therefore not an anomaly, but the discovery of a novel application for aptamers that is likely to have broad impacts on public health. FIG. 8 illustrates hyper-stimulatory behavioral phenotype caused by hexavalent chromium exposure is prevented by chromium-binding aptamer. L1-stage worms were plated to N2 plates, with or without 3.5 mM Cr(VI) as indicated, and with or without 100 μM chromium aptamer or scramble control. 10 mM copper chloride was applied to the animal as a noxious stimulus, and an aversive response was recorded (reverse movement away from the stimulus). Solvent controls (left bar, water only) were included for each trial condition and produced a minimal aversive response. n=6 experimental replicates, 10 worms/plate×3 plates per condition were tested within each experimental replicate.

### Environmental Pollutants:

Configurations herein employ aptamers having specificity for PFOA and bisphenol A.

A particular aptamer amendable to PFOA has been referred to as PFOA_JYP_2. Park, Environment International, doi.org/10.1016/j.envint.2021.107000 (2021), one of several related sequences and having a form of:

(SEQ ID NO. 6)
GGCGTGGGGTGGTAGGCTGTAAAGGGGGTC.

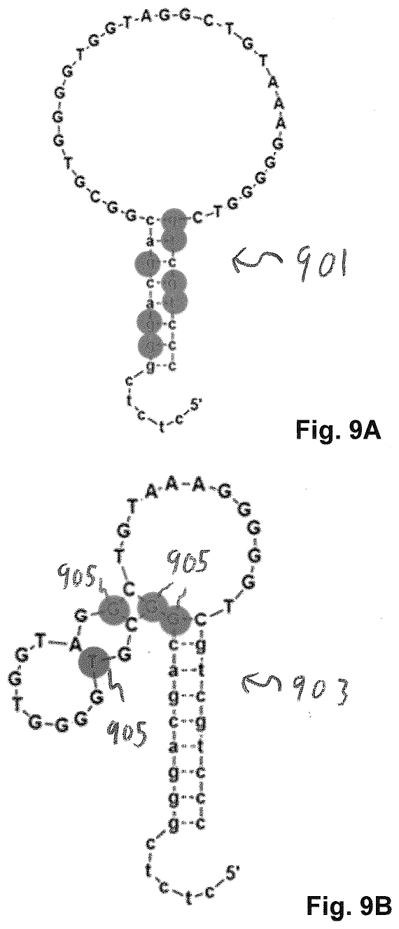

FIGS. 9A and 9B show the binding aptamer PFOA_JYP_2 used for sequester of PFOA and the predicted structure of an aptamer that binds PFOA, in the absence (FIG. 9A) and presence (FIG. 9B) of PFOA. FIG. 9A shows the secondary structure of the binding aptamer 901 in 0 mM PFOA at 5° C., and FIG. 9B shows the binding aptamer 903 in 0.5 mM PFOA at 5° C., and. When PFOA is present, the aptamer binds to the PFOA at the nucleotide sites 905 shown shaded in FIG. 9B. This aptamer was conventionally designed as a sensor to detect PFOA in a water sample

Using a selected aptamer corresponding to a particular pollutant, configurations herein depict a method of locating and removing pollutants by identifying a target pollutant sought for removal, and determining a binding aptamer having an affinity for the target pollutant. The selection of a proper corresponding aptamer can be applied towards a variety of pollutants or other substances sought for sequestration and removal or isolation. From the binding aptamer, a nucleic acid strand is generated including the binding aptamer. Based on the context or location of the pollutant contamination, the nucleic acid strand is introduced into a treatment region for binding with the target pollutant, whereby the bound target pollutant is configured for isolation and removal. As in FIG. 9B above, the aptamer binds to the target pollutant based on a correspondence of nucleotide sites between the binding aptamer and the target pollutant.

In the case of human or animal pollutant scenarios, the approach then is to form or generate the binding aptamer and either
1) inject them (as modified nucleic acids) into individuals to bind a toxin and eliminate it from the body,
2) to express these as short RNA molecules within an engineered probiotic bacterial strain to be used as a probiotic in the gastrointestinal tract to absorb and eliminate a toxin and prevent it from being absorbed by the body, or
3) to affix aptamers to a surface to be used as a filtration or elimination device for a toxin.

Therefore, implementing a remedial treatment may involve modifying a nucleic acid to include the binding aptamer in a modified nucleic acid, and introducing the modified nucleic acid into the treatment region for binding with the target pollutant.

A more robust approach having a greater duration may involve expressing the binding aptamer as an RNA molecule within an engineered bacterial strain, and establishing the bacterial strain in the treatment region. This effectively establishes an aptamer “factory” of cells for generating the binding aptamer. One approach involves modifying a cell for inclusion of the binding aptamer in a DNA or RNA structure in the cell by attaching a lipid to the nucleic acid strand to form a modified nucleic acid strand and depositing the modified nucleic acid strand in the cell.

Further, the binding aptamer could be adhering to a filtration surface in communication with the treatment region, such as an aqueous flow or recirculating fluid, and forming a flow engaging the filtration surface with the treatment region. Following a period of aptamer communication with the filtration surface in the flow, extraction of the filtration surface including the bound target pollutant removes the new sequestered pollutants.

Conventional approaches do not employ prophylactic or therapeutic strategy that an individual could use to rid themselves of a toxic organic pollutant, where prevention and avoidance are the paramount strategy. In use cases, binding aptamers could be applied as a prescribed therapeutic, however could also be employed in a bioengineered probiotic for over-the-counter use. Consumers are increasingly concerned about contaminants such as PFAS and bisphenols in their food, water, and air, and would be interested in strategies for remediating these exposures.

Modified RNAs are increasingly being used as prophylactics and therapeutics in the form of mRNA vaccines, antisense oligonucleotides (ASOs), and small interfering (si) RNAs, and their impact is expected to increase rapidly and significantly. In one configuration, aptamers that specifically bind an individual contaminant or a structurally-related class of contaminants could be injected into the bloodstream or tissues in a manner similar to current ASO and siRNA therapeutics, where it would circulate and bind to the target pollutants. Aptamers are known from human studies to be generally safe and efficiently excreted by the kidneys, which is a drawback for ASOs or siRNAs used for diseases, but in the context of eliminating toxins may be an asset as the toxin would be efficiently eliminated with the aptamer in the kidney.

In contrast to conventional use of aptamers for antibodies or therapeutic treatment, the purpose of the binding aptamer is to sequester and remove the target pollutant from the environment or organism. Conventional approaches seek to maintain and proliferate the aptamer around the organism, for combating pathogens, preventing binding with cell sites, and other purposes favoring longevity, not removal, of the bound aptamer.

Given the field of aptamers that are known and/or generated, coupled with pollutants or toxins that may define the target pollutant, effectiveness of pollutant removal is based on a correlation or mapping of the aptamer to the target pollutant and assuring a sufficient affinity to effect sequestration and removal. A significant step is in selecting the binding aptamer based on an ability to extract the bound target pollutant from the treatment region. Determining the binding aptamer further comprises identifying characteristics of the bound target pollutant, the characteristics including one or more of:
i: a bioavailability in the treatment region;
ii: a renal clearance for voiding the bound target pollutant; or
iii: non-activation of an immune system by the bound target pollutant.

As mentioned above, extraction from the treatment region completes the removal. Eradication from an organism may typically, and least invasively, be achieved via the natural processes of kidney and gastrointestinal removal. Thus, renal clearance is important to ensure the bound aptamer does not bypass the kidneys. Similarly, a non-activation of the host immune system avoids adverse side effects.

Identification a removal medium configured to convey the bound target pollutant away from the treatment region is important for validation. Such a removal medium selectively directs the bound target pollutant from the treatment region, particularly when the treatment region is a tissue area of a living organism. Validation of the presence of the target pollutant in the removal medium may be established by
i: a shape change of a physical strand defining the bound target pollutant,
ii: decay kinetics of the bound target pollutant, or
iii: sequestration of the bound target pollutant in an aqueous stream from the treatment.

Symptomatic results through observation may also be achieved. Referring again to FIGS. 9A and 9B, depending on binding sites 905 of the binding aptamer, a secondary shape may be observed once bound with the target pollutant.

In yet another configuration, the treatment region could be a gastrointestinal tract of a subject organism. In such a configuration, probiotic cells could be engineered to express aptamers that would collect, sequester, and eliminate ingested toxins. These probiotic organisms would collect the pollutants in the gastrointestinal tract and they would be eliminated along with solid (fecal) waste. Such a strategy would reduce absorption of toxins from food or water exposures, and decrease the risk of the associated disease burden of exposure.

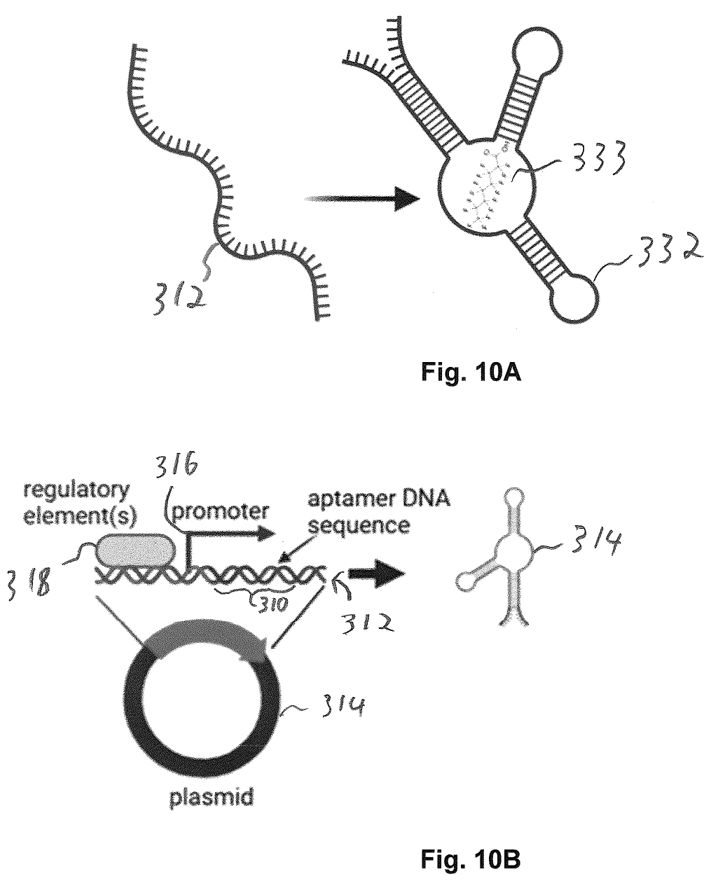

FIGS. 10A-10C show encoding or expressing the binding aptamer in a nucleic acid strand. Referring to FIGS. 9-10C, FIG. 10A is an example of a nucleic acid strand 312 including the binding aptamer 310 binding to an organic pollutant 333 such as PFOA. The binding aptamer 310 has been determined to have an affinity for the target is used to sequester the target pollutant. The aptamer may fold itself around the BPA molecule and sequester it from the cell or organism.

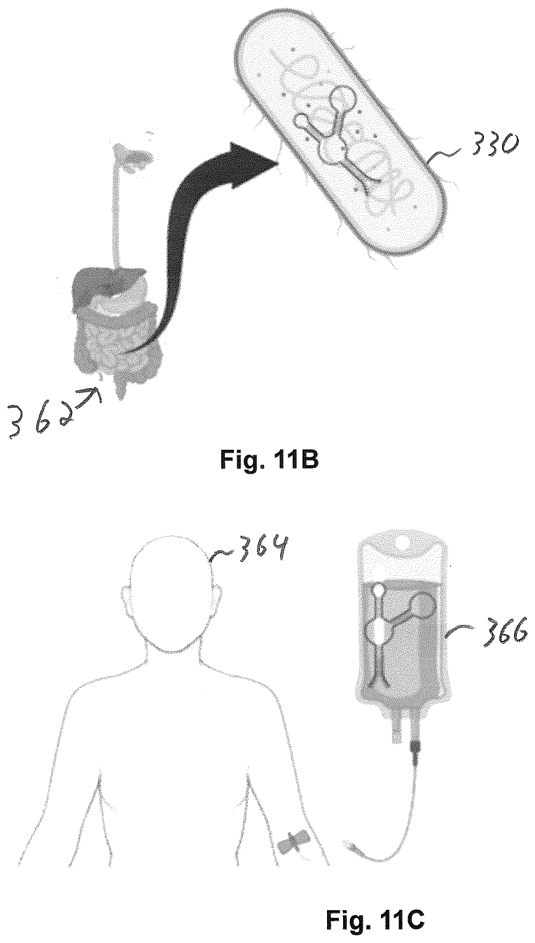

FIG. 10B shows generation of a nucleic acid strand 312 including the binding aptamer 310. A plasmid 314 is used for introducing the nucleic acid strand 312 into a cellular organism 330 for binding with the target pollutant 333. The target pollutant 333 may be any suitable molecule or compound of earthly origin, or connected to the natural world and human existence, however need not be essential to the physiology of the cellular organism. The cellular organism is allowed to thrive, interact and reproduce within the region containing the target pollutant, often an affected human or animal manifesting the target pollutant 333, such as PFOA 333′, to allow for absorption of the target pollutant into the cellular organism. Following binding of the nucleic acid strand 312 with the target pollutant 333 as a bound aptamer 332, separation of the target pollutant for collection occurs, discussed below with respect to FIGS. 12A and 12B.

Once the binding aptamer having an affinity for the target pollutant 333 is identified and/or found, the binding aptamer 310 is encoded onto a plasmid 314. The binding aptamer may be expressed by a promoter 316 selected based on the cellular organism. The binding aptamer 310 may also be expressed using a regulatory element 318. In the example of FIG. 10B, the aptamer 310 sequence with an affinity for the target pollutant 333 will be encoded on a plasmid, and the plasmid 314 inserted into the cellular organism 330. Expression of the aptamer sequence 310 will be driven by a promoter 316 appropriate for the target organism and may be controlled by additional regulatory elements. Expression of the genetic construct therefore results in the production of an RNA version of a nucleic acid strand 312 of the binding aptamer 310 within the cell.

The cellular organism 330 is now exhibiting the binding aptamer 310 and can effectively carry and reproduce additional cells for propagating the binding aptamer throughout affected human or animal. The cellular organism 330 absorbs the target pollutant 333 for sequestration within the cellular organism 330. In FIG. 10C, In the case of a probiotic cell as the cellular organism 330, the aptamer 310 would be expressed inside of a cell from a plasmid containing the aptamer sequence and an appropriate promoter for expression in the specific organism.

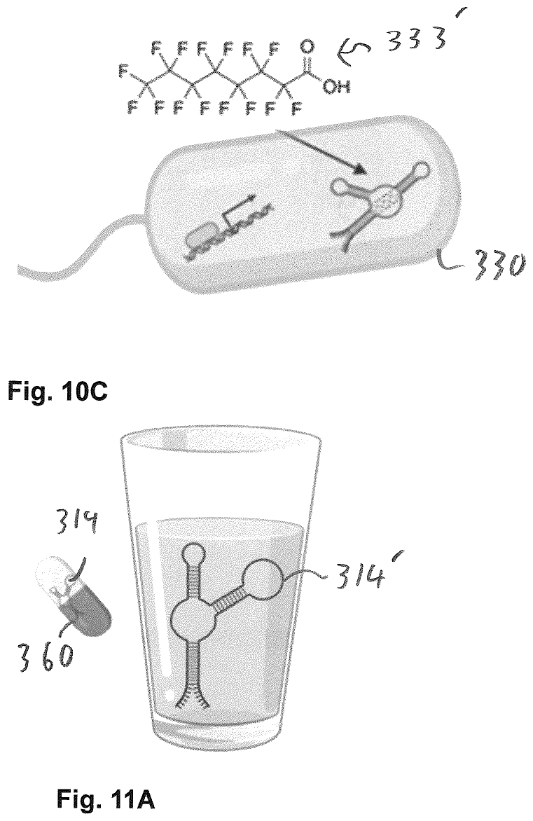

FIGS. 11A-11C show approaches for introducing the nucleic acid strand including the binding aptamer into a treatment region. Referring to FIGS. 9-11C, various prophylactic and therapeutic applications of aptamers 310 may be used to protect individuals from target pollutants 333. In FIG. 11A, aptamers 310 could simply be added to water as a quick, point of use treatment, possibly by an ingested capsule. A pollutant bound to an aptamer and then ingested would not be absorbed by the gastrointestinal tract and would be eliminated in the waste. In FIG. 11B, genetically engineered probiotic bacteria could express the aptamer 310 and then be ingested to colonize the gut. When target pollutants 333 are later ingested, these engineered probiotic strains would absorb and sequester the pollutants, and eventually be eliminated in the waste of the affected human or animal. In FIG. 11C, for treatment of acute poisoning with pollutant substances, the binding aptamer 310 can be injected or infused via an IV bag into a patient and would circulate in the body fluids and absorb the target pollutant 333. Aptamers and their bound pollutants are then efficiently excreted via the renal system.

FIGS. 12A-12B show extraction of the target pollutant 333 from the bound aptamer and delivery substance or organism. In FIG. 12A, In the case of a probiotic cell 330, the binding aptamer 310 would be expressed inside of a cell from the plasmid 314 containing the aptamer strand 312 and an appropriate promoter for expression in the specific organism. In the case of a filtration device, as in FIG. 12B, the binding aptamer 310 may be tethered to a filtration surface 350 for the purpose of retaining the pollutant in the filter via an aqueous stream 352.

In one example, generation of an aptamer transfection assay for a binding aptamer 310 targeting BPA as the target pollutant 333 is as follows:

(SEQ ID NO. 7)
(5'-
CCGGTGGGTGGTCAGGTGGGATAGCGTTCCGCGTATGGCCCAGCGCAT
CACGGGTTCGCACCA-3'),

or a scrambled sequence control

(SEQ ID NO. 8)
(5'-
GGGCGGCGCTGGTCCGGGCTGTTCCCGCCCAAGATCGTGAGATAGGCA
GACTTTTGCCTGAGG-3'),

### Or:

BPA aptamer #2:

(SEQ ID NO. 9)
5'-
GGGCAACTCCAAGCTAGATCTACCGGTGTATTTAGTAAGCACACGGTA
ATGCGACTGGGCAAGTCTTCTACTGGCTTCTACAGCGCCCTTAAAATGG
CTAGCAAAGGAGAAGAACTTTTC-3'

at a final concentration of 34 nM (nanomolar) is complexed into liposomes for transfection using commercial lipid-based transfection reagents (e.g. ThermoFisher® Lipofectamine® 3000) and applied to cultured human cervical cancer cells (HeLa) in culture medium. A mock transfection consisting of empty liposomes is also performed. After 24 hours, the culture medium is replaced with a medium containing bisphenol A (BPA) at 10 nM, or medium without BPA as a control, and incubated for an additional 48 hours. Then the cells are harvested and RNA is extracted. The RNA is reverse transcribed into cDNA (complementary DNA) in a reverse transcriptase reaction. The cDNA is used in a quantitative PCR reaction to measure the relative abundance of specific mRNAs for particular genes. The BRCA1 gene is known to increase in response to exposure of cells to BPA, and the GMNN gene was found to decrease slightly in response to BPA.

A similar process for PFOA involves the following form of PFOA_JYP_2:

(SEQ ID NO. 10)
5'-
CTCTCGGGACGACGGCGTGGGGTGGTAGGCTGTAAAGGGGGTCGTCGT
CCC-3'

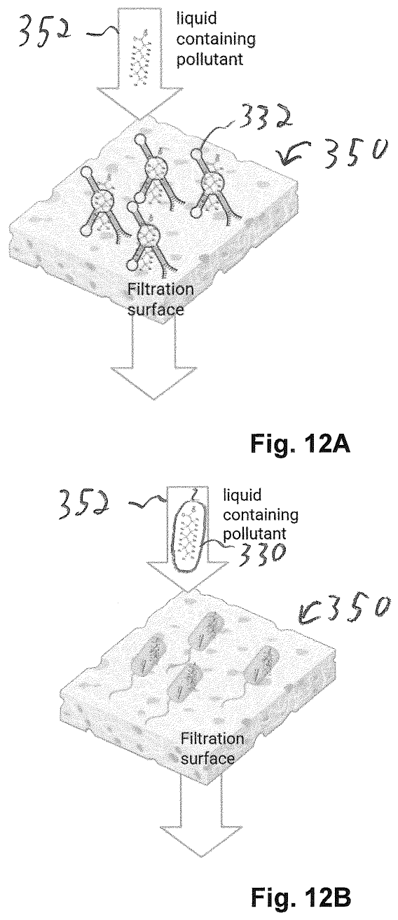

FIG. 13 shows qPCR (quantitative polymerase chain reaction) analysis of a binding aptamer expression in response to bisphenol A. Referring to FIG. 13, the untreated control is not exposed to liposomes and not exposed to BPA, and is used as a reference for gene expression against which all other conditions are normalized. 10 nM BPA exposure in a mock transfection condition (mock+BPA 10 nM) results in an approximately 1.4-fold increase in expression of BRCA1 gene mRNA. Transfection with a scrambled aptamer sequence which does not target the BPA, combined with 10 nM BPA exposure (scramble+BPA 10 nM), results in a similar increase in expression of BRCA1 mRNA relative to the untreated control. However, transfection of the aptamer in combination with BPA (aptamer+BPA 10 nM) results in BRCA1 mRNA expression that is similar to the untreated control. Thus, the aptamer prevents the change in BRCA1 gene expression caused by BPA, thereby protecting the cell from effects of BPA.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of locating and removing pollutants, comprising:
identifying a target pollutant sought for removal;
determining a binding aptamer having an affinity for the target pollutant;
generating a nucleic acid strand including the binding aptamer; and
introducing the nucleic acid strand into a treatment region for binding with the target pollutant, whereby the bound target pollutant is configured for isolation and removal.

2. The method of claim 1 further comprising binding the binding aptamer to the target pollutant based on a correspondence of nucleotide sites between the binding aptamer and the target pollutant.

3. The method of claim 1 further comprising:
modifying a nucleic acid to include the binding aptamer in a modified nucleic acid; and
introducing the modified nucleic acid into the treatment region for binding with the target pollutant.

4. The method of claim 1 further comprising:
expressing the binding aptamer as an RNA molecule within an engineered bacterial strain;
establishing the bacterial strain in the treatment region.

5. The method of claim 1 further comprising:
adhering the binding aptamer to a filtration surface in communication with the treatment region;
forming a flow engaging the filtration surface with the treatment region; and
extracting the filtration surface including the bound target pollutant.

6. The method of claim 4 wherein treatment region is a gastrointestinal tract of a subject organism.

7. The method of claim 1 further comprising:
selecting the binding aptamer based on an ability to extract the bound target pollutant from the treatment region.

8. The method of claim 7 wherein determining the binding aptamer further comprises identifying characteristics of the bound target pollutant, the characteristics including one or more of:
i: a bioavailability in the treatment region;
ii: a renal clearance for voiding the bound target pollutant; or
iii: non-activation of an immune system by the bound target pollutant.

9. The method of claim 1 further comprising:
identifying a removal medium configured to convey the bound target pollutant away from the treatment region; and
selectively directing the bound target pollutant from the treatment region.

10. The method of claim 1 wherein the treatment region is a tissue area of a living organism.

11. The method of claim 4 further comprising
modifying a cell for inclusion of the binding aptamer in a DNA or RNA structure in the cell by attaching a lipid to the nucleic acid strand to form a modified nucleic acid strand and depositing the modified nucleic acid strand in the cell.

12. The method of claim 9 further comprising validating the presence of the target pollutant in the removal medium by one or more of:
i: a shape change of a physical strand defining the bound target pollutant,
ii: decay kinetics of the bound target pollutant, or
iii: sequestration of the bound target pollutant in an aqueous stream from the treatment region.


SEQUENCE LISTING (10 sequences)

SEQ ID NO: 1 - RNA, 76 nt, Synthetic construct
  source 1..76: mol_type = other RNA
ggaggctctcgggacgacggcagggctgtcgtacggtttgtcgaaggtgtcgtcccgatgctgcaatcgtaagaat

SEQ ID NO: 2 - RNA, 48 nt, Synthetic construct
  source 1..48: mol_type = other RNA
gggacgacggcagggctgtcgtacggtttgtcgaaggtgtcgtcccga

SEQ ID NO: 3 - RNA, 48 nt, Synthetic construct
  source 1..48: mol_type = other RNA
tcgggacgacaccttcgacaaaccgtacgacagccctgccgtcgtccc

SEQ ID NO: 4 - RNA, 48 nt, Synthetic construct
  source 1..48: mol_type = other RNA
gcgggcgatctgcggacgttctgagcctgactgagtggggacgctgta

SEQ ID NO: 5 - RNA, 62 nt, Synthetic construct
  source 1..62: mol_type = other RNA
ccacgcatagggcaaatcaagcacaccctctaatgttgcctctgattctggcctatgcgtgc

SEQ ID NO: 6 - RNA, 30 nt, Synthetic construct
  source 1..30: mol_type = other RNA
ggcgtggggtggtaggctgtaaagggggtc

SEQ ID NO: 7 - RNA, 63 nt, Synthetic construct
  source 1..63: mol_type = other RNA
ccggtgggtggtcaggtgggatagcgttccgcgtatggcccagcgcatcacgggttcgcacca

SEQ ID NO: 8 - RNA, 63 nt, Synthetic construct
  source 1..63: mol_type = other RNA
gggcggcgctggtccgggctgttcccgcccaagatcgtgagataggcagacttttgcctgagg

SEQ ID NO: 9 - RNA, 120 nt, Synthetic construct
  source 1..120: mol_type = other RNA
gggcaactccaagctagatctaccggtgtatttagtaagcacacggtaatgcgactgggcaagtcttctactggcttctacagcgcccttaaaatggctagcaaaggagaagaacttttc

SEQ ID NO: 10 - RNA, 51 nt, Synthetic construct
  source 1..51: mol_type = other RNA
ctctcgggacgacggcgtggggtggtaggctgtaaagggggtcgtcgtccc